(19) United States
(12) Patent Application Publication
(10) Pub. No.: US 2021/0003568 A1
(43) Pub. Date: Jan. 7, 2021

(54) NOVEL PREGNANCY DIAGNOSIS DEVICE INCLUDING BETA-CORE FRAGMENT HCG AS MARKER

(71) Applicant: ADTECH CO., LTD., Gunpo-si (KR)

(72) Inventors: Jin Dong CHANG, Seoul (KR); Sung Jin CHO, Cheonan-si (KR)

(73) Assignee: ADTECH CO., LTD., Gunpo-si (KR)

(21) Appl. No.: 16/572,364

(22) Filed: Sep. 16, 2019

Related U.S. Application Data
(63) Continuation of application No. PCT/KR2019/009573, filed on Jul. 31, 2019.

(30) Foreign Application Priority Data
Jul. 4, 2019 (KR) 10-2019-0080618

(51) Int. Cl. G01N 33/543; G01N 33/76

(52) U.S. Cl. CPC G01N 33/54386; G01N 2333/471; G01N 33/76

(57) ABSTRACT

An immune device for pregnancy diagnosis of the inventive concept includes a sample region for receiving a test sample to be analyzed; a conjugate region connected to the sample region and including an anti-I-hCG antibody conjugated with a probe and an anti-βcf hCG antibody conjugated with a probe; a signal detection region connected with the conjugate region, wherein the signal detection region includes a first test line having an anti-I-hCG antibody immobilized to the first test line, a second test line having an anti-βcf hCG antibody immobilized to the second test line, and a control line; and a wicking region located downstream of the signal detection region, wherein the wicking region absorbs the test sample for which a signal detection reaction has terminated.

### CROSS-REFERENCE TO RELATED APPLICATIONS

The present application is a continuation of International Patent Application No. PCT/KR2019/009573, filed Jul. 31, 2019, which is based upon and claims the benefit of priority to Korean Patent Application No. 10-2019-0080618, filed on Jul. 4, 2019. The disclosures of the above-listed applications are hereby incorporated by reference herein in their entirety.

### BACKGROUND

Embodiments of the inventive concept described herein relate to an immune device for diagnosing pregnancy, and a pregnancy diagnosis method using the same.

Human chorionic gonadotropin (hCG) is a glycoprotein hormone produced during pregnancy. hCG is produced in a chorionic trophoblast of placenta and continues to produce progesterone in an early stage of pregnancy, thus maintaining implantation until 10 weeks of pregnancy when the placental function is completed. Intact hCG (I-hCG) in an active form in a body has a molecular weight of about 37 kDa and is composed of 244 amino acids. I-hCG is composed of two subunits, that is, alpha (α) subunit and beta (β) subunit. The alpha subunit contains 92 amino acids and the beta subunit contains 145 amino acids. I-hCG has a main structure that usually exists at various stages of pregnancy and exists in the body. In the early stage of pregnancy, this active structure is present at a high percentage in a form of hyperglycosylated hCG (H-hCG). In addition to the active form, various dissociation and degradation products of hCG are found in blood and urine. These products include nicked hCG (N-hCG), free beta hCG (free β-hCG), free alpha hCG (free α-hCG), free beta-core fragment hCG (free β-core fragment hCG), etc.

The beta-core fragment hCG (βcf hCG) refers to a fragment of free β-hCG, as one distinct degradation product of hCG and has a molecular weight of about 14 kDa. The βcf hCG begins to be detected in urine at about 0.01 pmol/mL smaller compared to a content of I-hCG. The content of the βcf hCG gradually increases as the number of gestational weeks increases. Then, after 5 weeks thereof, the content thereof is larger than that of I-hCG. In addition, unlike other metabolites, the βcf hCG is present only in urine (J Clin Endocrinol Metab, 76, 1993, 704-10).

An I-hCG concentration is highest in serum and urine in a period between 9 to 12 weeks. A concentration thereof in urine of a pregnant woman is 150,000 mIU/mL at 10 weeks. Depending on individual differences, a high concentration of 900,000 mIU/mL of the I-hCG may be detected in urine. Currently used pregnancy diagnostic kits qualitatively diagnose the I-hCG at the early stage of pregnancy (3 to 5 weeks from a last menstrual date) to determine pregnancy or non-pregnancy. However, when the existing pregnancy diagnosis kits were used for 11,760 people who were determined to be pregnant, about 0.2% thereof, that is, 22 people were found to be false negative. In this connection, I-hCG concentrations of these 22 people as measured have a maximum of 268,022.5 IU/mL (J. Emergency Medicine 2013, 44: 155). This indicates that the current pregnancy diagnostic kits are experiencing false negative problems due to a hook effect.

The false negative in pregnancy diagnosis may have serious consequences in emergency department (ED) situations. For example, even when the female patient is pregnant, the false negative result may occur when measuring urine using a general pregnancy diagnostic kit. In this case, the female patient may be treated without considering the fetus, thereby leading to a medical accident. That is, the kit does not diagnose an ectopic pregnancy, or drugs prohibited during pregnancy may be applied to the female patient, or radiation may be exposed to the fetus, thus leading to lawsuit. MAUDE (Manufacturer and user facility device experience database) investigation results by FDA have a case in which a pregnancy qualitative test of a female patient shows a negative result, and thus the patient is subjected to cervical biopsy and then I-hCG is detected in a serum test, a case in which a female patient is determined to be negative in a pregnancy test using urine and then CT was performed and then the patient has 8 weeks gestation period, and a case in which a female patient is determined to be negative in a pregnancy test and thus an intrauterine contraceptive device is installed or surgical operation is performed, but then pregnancy was determined in a later test (Nerenz et al., Clinical Chemistry 61: 3, 483-486, 2015).

The pregnancy diagnostic kit using urine generally detects I-hCG in a test line using an antibody against I-hCG as a label. However, following cases may result in false negatives in pregnancy diagnosis: when presence of a large amount of I-hCG in urine saturates a detection antibody immobilized on a diagnostic kit membrane and an antibody conjugated with a probe-marker such as gold or fluorescence label to prevent sandwich formation in the I-hCG test line of the diagnostic kit (in other words, the hook phenomenon may result in a false negative), when Bcf hCG is present in a high concentration in the urine, so that a free floating I-hCG antibody conjugated with a probe is coupled to βcf hCG to interfere with the binding thereof to the I-hCG, such that even though the I-hCG binds to the detection antibody immobilized on the test line of the diagnostic kit, the free floating I-hCG antibodies bound to βcf hCG do not bind to the I-hCG as an antigen, thereby to prevent sandwich formation (Griffey et al., The journal of Emergency Medicine, 44, 2013, 155-160).

In other words, it has been reported that the pregnancy diagnostic kit using urine may have a false negative result in pregnancy diagnosis due to a hook phenomenon when the I-hCG has a high concentration, and, further, a high concentration of βcf hCG may result in false negatives (Diwan, Med. Lab. Obeserver, 2011, 18-20; Cervinski et al., Clin. Chem. Lab. Med. 48(7), 2010, 935-942; Gronowski et al., Clin. Chem., 55(7), 2009, 1389-1394). Therefore, about two weeks after the last menstrual period (LMP), the concentration of βcf hCG increases, such that an interference effect to interfere with the sandwich formation of I-hCG may result in false negative.

Most of the previous pregnancy diagnostic kits detect only I-hCG. To solve the false negative problem in the diagnosis, some pregnancy diagnostic kits detect combinations of hCG-H and hCG-βin addition to I-hCG. Thus, false negatives due to the hook phenomenon resulting from the high concentration of I-hCG or false negatives due to suppression of normal sandwich formation by βcf hCG could be caused. Further, a kit that simultaneously detect I-hCG and βcf hCG and other types of hCG hormones in one test line exhibits reactivity to βcf hCG. However, this kit results in a false negative result due to a mutual interference effect in one test line for a sample having a mixture between I-hCG and βcf hCG at the high concentrations. For example, a pregnancy diagnostic kit from a C&D company includes an antibody that detects βcf hCG and other types of mutated hCG in addition to I-hCG in one test line. However, the mutual interference effect reduces the sensitivity in the test line, thus resulting in a false negative result.

Thus, the present inventors are continuing to research to solve the problems of the prior art related to the pregnancy diagnostic kit. Then, we have invented a pregnancy diagnostic kit in which a test line for detecting β-cf hCG is added in addition to a test line for detecting I-hCG, and each of I-hCG antibody and βcf hCG antibody free of mutual interference is disposed such that even when the hook phenomenon occurs due to the high concentration of I-hCG, the kit may accurately diagnose pregnancy based on a color rendering of the βcf hCG test line, thereby to minimize the false negative result.

### SUMMARY

Embodiments of the inventive concept provide an immune device and a pregnancy diagnosis method having an accurate pregnancy result, in which a second test line for detecting βcf hCG is added in addition to a first test line for detecting I-hCG, thereby minimizing the false negative result due to the hook phenomenon which was the problem of the prior art related to the pregnancy diagnostic kit, and in which even when the high concentration of I-hCG leads to the hook phenomenon, the pregnancy can be diagnosed by detecting βcf hCG and further the false negative due to the interference by the βcf hCG may be minimized.

According to an exemplary embodiment, the inventive concept provides a pregnancy diagnostic kit including an antibody to specifically recognize βcf hCG.

According to an exemplary embodiment, an immune device for pregnancy diagnosis may include a sample region for receiving a test sample to be analyzed; a conjugate region connected to the sample region and including an anti-I-hCG antibody conjugated with a probe and an anti-βcf hCG antibody conjugated with a probe; a signal detection region connected with the conjugate region, which is connected with the sample region, wherein the signal detection region includes a first test line having an anti-I-hCG antibody immobilized to the first test line, a second test line having an anti-βcf hCG antibody immobilized to the second test line, and a control line; and a wicking region located downstream of the signal detection region, wherein the wicking region absorbs the test sample for which a signal detection reaction has terminated.

According to one implementation of the inventive concept, the probe may include at least one selected from a group consisting of gold nanoparticles, silver nanoparticles, quantum dot nanoparticles, carbon nanoparticles, latex beads/fluorescent nanoparticles, cellulose nanoparticles, magnetic nanoparticles, silica nanoparticles, polymer beads, a fluorescent substance (fluorescein), a luminescent substance, a dye, and a protein.

According to one implementation of the inventive concept, the signal detection region may include a first test line having an anti-I-hCG antibody immobilized thereto, a second test line downstream of the first test line and having an anti-βcf hCG antibody immobilized thereto, and a control line downstream of the second test line. According to another implementation of the inventive concept, the signal detection region may include a second test line having an anti-βcf hCG antibody immobilized thereto, a first test line having an anti-I-hCG antibody immobilized thereto and downstream of the second test line, and a control line downstream of the first test line.

According to one implementation of the inventive concept, the signal detection region may include one selected from a group consisting of nitrocellulose, cellulose, polyethylene, polyethersulfone, and nylon.

According to one implementation of the inventive concept, the wicking region may nclude a porous support and an absorbent dispersed in pores of the porous support or adsorbed or coated on a fiber of the porous support.

According to one implementation of the inventive concept, the immune device may determine that a pregnancy is present when the control line and the first test line, the control line and the second test line or the control line and the first and second test lines emit a color.

According to an exemplary embodiment, a method for providing information for pregnancy diagnosis may include applying a test sample to the immune device, such that I-hCG and βcf hCG in the sample react with the anti-I-hCG antibody conjugated with the probe and the anti-βcf hCG antibody conjugated with the probe; checking the reaction of the I-hCG and βcf hCG in the sample using the first test line and the second test line, wherein presence of each of the I-hCG and βcf hCG is detected based on a signal resulting from presence of a sandwich complex in each of the first and second test lines; and determining that a pregnancy is present when the control line and the first test line, the control line and the second test line or the control line and the first and second test lines emit a color.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a schematic diagram showing an embodiment of an immunochromatography strip prepared according to the inventive concept.

FIG. 2 is a model diagram showing an embodiment in which the immunochromatography strip is applied to a diagnostic kit according to the inventive concept. 1 denotes a readout window of the diagnostic kit; 2 denotes a control line indicating an end of a test; 3 and 4 denote test lines; and 5 denotes a sample region into which a test sample is applied.

FIG. 3 shows an aspect of first and second test lines and a control line of the kit including the immunochromatography strip prepared according to the inventive concept based on I-hCG and βcf hCG concentrations.

FIG. 4 shows a βcf hCG response evaluation result of the kit including the immunochromatography strip of the inventive concept compared to a pregnancy diagnostic kit from a third-party company.

FIG. 5 shows a comparison result in which when a female patient is determined to false negative using a conventional pregnancy immunochromatography strip, whereas the female patient is determined to be positive using the immunochromatography strip with heterogeneous test lines of the present concept.

FIG. 6 shows a comparison result between Comparative Example 1 (C&D's pregnancy diagnostic kit) and a pregnancy diagnostic kit of the inventive concept.

### DETAILED DESCRIPTION

Advantages and features of the inventive concept and methods of achieving them will become apparent with reference to the embodiments described below in detail with the accompanying drawings. However, the inventive concept is not limited to the embodiments disclosed below, but may be implemented in various different forms. The present embodiments merely allow the disclosure of the inventive concept to be complete, and completely inform the scope of the inventive concept to those of ordinary skill in the technical field to which the inventive concept belongs. The inventive concept is only defined by the scope of the claims. Details for the implementation of the inventive concept will be described with reference to the accompanying drawings. The same reference numbers in different figures denote the same or similar elements, and as such perform similar functionality. As used herein, the term “and/or” includes any and all combinations of one or more of the associated listed items.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, an immune device for diagnosing pregnancy and a pregnancy diagnosis method using the same according to embodiments of the inventive concept will be described.

An immune device for diagnosing pregnancy according to embodiments of the inventive concept includes: a sample region for receiving a test sample to be analyzed; a conjugate region connected to the sample region and including an anti-I-hCG antibody conjugated with a probe and an anti-βcf hCG antibody conjugated with a probe; a signal detection region connected with the conjugate region, which is connected with the sample region, wherein the signal detection region includes a first test line having an anti-I-hCG antibody immobilized to the first test line, a second test line having an anti-βcf hCG antibody immobilized to the second test line, and a control line; and a wicking region located downstream of the signal detection region, wherein the wicking region absorbs the test sample for which a signal detection reaction has terminated.

The sample region preferably receives a liquid sample, such as blood or urine, of females of childbearing age. However, any sample expected to include I-hCG and/or βcf hCG may be received into the sample region.

The sample region may further have a function of filtering to further improve a selectivity to an analyte or to minimize an effect of interfering substances that may be included in the sample. If necessary, the device may further include an additional region upstream of the sample region. The additional region includes a substance that can increase reaction between the analyte and the conjugate or exclude the effect of the interfering substance.

The control line of the conjugate region refers to a portion that produces a constant signal regardless of the concentration of I-hCG or βcf hCG in the test sample. The control line may be formed by immobilizing a ligand thereto that binds to a third conjugate moving along the detection region in a mobile phase with the test sample without binding to the I-hCG or βcf hCG. The control line is configured to immobilize a ligand thereto that can emit a constant signal regardless of presence or absence of I-hCG and βcf hCG in the sample. Therefore, even when a first conjugate and/or a second conjugate is not combined with a target sample and thus passes by the first test line and/or the second test line, the third conjugate may be combined with the sample and captured by the control line. For example, when a target sample does not exist in the sample, a color may be rendered in the control line even though the color is not rendered in the test line. As such, a response in the control line means that the liquid sample is properly passing through the immune device. The third conjugate may include, for example, mouse IgG or chicken IgY. The control line may contain anti-rabbit IgG, anti-chicken IgY, streptavidin, bovine serum albumin, goat anti-mouse IgG or goat-anti-chicken IgY.

The probe in the conjugate region may include at least one selected from a group consisting of gold nanoparticles, silver nanoparticles, quantum dot nanoparticles, carbon nanoparticles, latex beads/fluorescent nanoparticles, cellulose nanoparticles, magnetic nanoparticles, silica nanoparticles, polymer beads, a fluorescent substance (fluorescein), a luminescent substance, a dye, and a protein. According to one implementation of the inventive concept, the probe may include colloidal gold nanoparticles but is not limited thereto.

According to one implementation of the inventive concept, the signal detection region may include a first test line having an anti-I-hCG antibody immobilized thereto, a second test line downstream of the first test line and having an anti-βcf hCG antibody immobilized thereto, and a control line downstream of the second test line. According to another implementation of the inventive concept, the signal detection region may include a second test line having an anti-βcf hCG antibody immobilized thereto, a first test line having an anti-I-hCG antibody immobilized thereto and downstream of the second test line, and a control line downstream of the first test line.

The signal detection region may include a medium in which the mobile phase and the sample move together. The mobile phase and the test sample may be moved by capillary phenomenon of a porous membrane in the signal detection region. The signal detection region may include one selected from a group consisting of nitrocellulose, PVDF, polyvinyl resin or a well plate synthesized with polystyrene resin, and a slide glass made of glass. According to one implementation of the inventive concept, the signal detection region may include nitrocellulose but is not limited thereto. According to one implementation of the inventive concept, a nitrocellulose membrane having pores of 5 to 15 μm may be used in the detection region but is not limited thereto.

The wicking region may include a porous support and an absorbent dispersed in pores of the porous support or adsorbed or coated on a fiber of the porous support. The wicking region may further include a porous film layer on a top face of the porous support, but is not limited thereto. The absorbent may be selected from a group consisting of calcium chloride, magnesium chloride, diatomaceous earth, bentonite, dolomite, gypsum, silica gel and mixtures thereof, but is not limited thereto.

In accordance with the inventive concept, a concentration measurement range of a target substance in the sample based on a signal strength of each of the first test line and the second test line is equal to or greater than 25 mIU/mL for I-hCG, and 1 pmol/mL for βcf hCG.

A conventional immunochromatography strip shows a phenomenon that a signal strength in the test line is reduced due to the occurrence of the hook phenomenon when a concentration of the target substance in the sample exceeds a certain concentration. However, in an immune device having the two test lines of the inventive concept, even when the concentration of I-hCG in the sample increases, a pregnancy may be detected using the second test line for detecting βcf hCG irrespective of the hook phenomenon caused by the increase in I-hCG concentration. Further, the immune device may include the anti-βcf hCG antibody in the test line to minimize the false negative due to the interference caused by increasing the βcf hCG concentration in the sample. Therefore, the immune device determines that the pregnancy is present when the control line and the first test line, the control line and the second test line or the control line and the first and second test lines on the immunochromatography strip exhibit a color. In this connection, in about 3 weeks of pregnancy, the conventional immunochromatography strip shows the false negative due to the interference caused by the increase of βcf hCG. However, unlike the conventional pregnancy diagnostic kit, in the immune device having the two test lines of the inventive concept, the presence of βcf hCG is detected by the separate test line having the anti-βcf hCG antibody to accurately diagnose the pregnancy. Therefore, the immune device having the two test lines of the inventive concept has the feature that the combination of the first test line and the second test line extends a range of the pregnancy diagnosis which has been limited due to a single test line of the prior art, and thus improves diagnosis accuracy.

In accordance with the inventive concept, a first conjugate of the anti-I-hCG antibody present in the conjugate region together with the liquid sample such as blood or urine of fertility women may move toward a membrane, and then may bind to the I-hCG in the sample to form a first I-hCG-conjugate complex. A second conjugate of the anti-βcf hCG antibody present in the conjugate region together with the liquid sample such as blood or urine of fertility women may move toward a membrane, and then may bind to the βcf hCG in the sample to form a second βcf hCG-conjugate complex. The first complex reacts with the anti-I-hCG antibody immobilized on the first test line during movement thereof, while the second complex reacts with the anti-βcf hCG antibody immobilized on the second test line during movement thereof. The signal strength of the I-hCG test line increases as the I-hCG concentration in the sample increases. However, when the concentration thereof increases to a level above a certain concentration, the signal strength decreases due to the hook phenomenon. The immunochromatography strip of the inventive concept may use the second test line to sensitively detect the βcf hCG even when a large amount of I-hCG causes the hook phenomenon. Thus, the immunochromatography strip of the inventive concept may detect that the pregnancy is present while not being affected by the hook phenomenon.

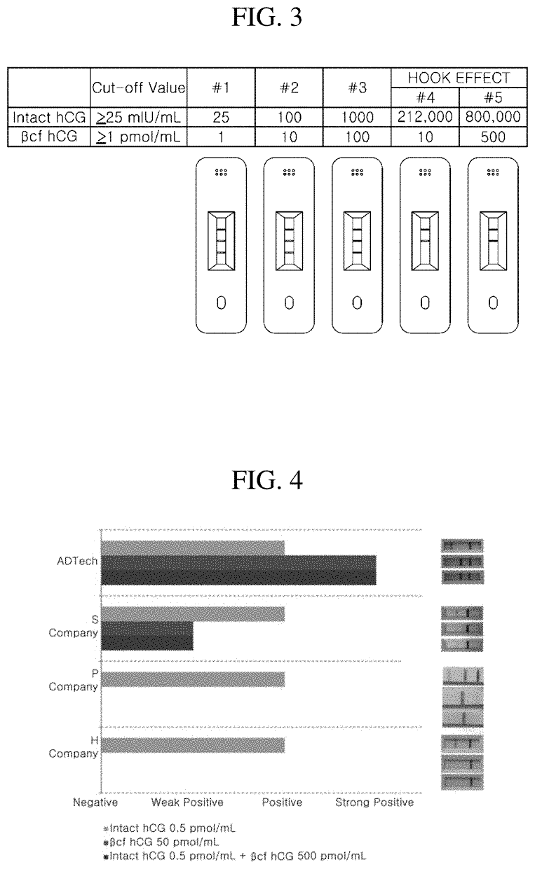

The first test line and the second test line results according to the I-hCG concentration increase of the inventive concept are shown in FIG. 3. The second test line detects the βcf hCG and diagnoses that the pregnancy is positive even in the concentration of I-hCG at which the hook phenomenon occurs.

Further, the immune device of the inventive concept may include a solid support as an lower portion.

The solid support may be formed of a material selected from a group consisting of nitrocellulose, nylon, PVDF, glass and plastic. The device may be manufactured by attaching the strip on the solid support. Thus, the strip may be more durable and easier to handle and store. Further, this may facilitate installation of an additional external device thereon.

The plastic material that may be used as a material of the solid support may include a polypropylene film, a polyester film, a polycarbonate film, an acrylic film, etc. but is not limited thereto.

Another aspect of the inventive concept discloses a kit in which the immune device is additionally fixed to another device. A lower device of the kit has a guide and strip support, and an upper device thereof has a sample inlet, and a result checking window exposing the first test line, the second test line, and the control line.

The upper and lower devices may be manufactured using conventional plastic materials. For example, a material such as polycarbonate and acrylonitrile butadiene styrene (ABS) may be used for the upper and lower devices. However, the inventive concept is not limited thereto.

According to one implementation of the inventive concept, the immunological device for pregnancy diagnosis of the inventive concept may determine that the pregnancy is positive when 1) the control line and the first test line, 2) the control line and the second test line, or 3) the control line, and the first test line and the second test line emit a color.

Another aspect of the inventive concept discloses a method for providing information for pregnancy diagnosis including applying a test sample to the immune device, such that I-hCG and βcf hCG in the sample react with the anti-I-hCG antibody conjugated with the probe and the anti-βcf hCG antibody conjugated with the probe; checking the reaction of the I-hCG and βcf hCG in the sample using the first test line and the second test line, wherein presence of each of the I-hCG and βcf hCG is detected based on a signal resulting from presence of a sandwich complex in each of the first and second test lines; and determining that a pregnancy is present when the control line and the first test line, the control line and the second test line or the control line and the first and second test lines emit a color.

Still another aspect of the inventive concept discloses a pregnancy diagnosis method including applying a test sample to the immune device, such that I-hCG and βcf hCG in the sample react with the anti-I-hCG antibody conjugated with the probe and the anti-βcf hCG antibody conjugated with the probe; checking the reaction of the I-hCG and βcf hCG in the sample using the first test line and the second test line, wherein presence of each of the I-hCG and βcf hCG is detected based on a signal resulting from presence of a sandwich complex in each of the first and second test lines; and determining that a pregnancy is present when the control line and the first test line, the control line and the second test line or the control line and the first and second test lines emit a color.

### PRESENT EXAMPLE

Hereinafter, the inventive concept will be described in detail with reference the Present Example.

However, the following Present Example is merely to illustrate the inventive concept, and contents of the inventive concept is not limited by the Present Example.

### Present Example 1

### Preparation of Immunochromatography Strip

A. Fabrication of membrane with First Test Line, Second Test Line, and Control Line

Three assay ligands were dispensed onto a membrane (nitrocellulose membrane). Anti-I-hCG monoclonal antibody (Monoclonal anti-hCG) as an I-hCG antibody of the first test line was dispensed and dried on the first test line. Anti-βcf hCG monoclonal antibody (Monoclonal anti-βcf hCG) as a βcf hCG antibody of the second test line was dispensed and dried on the second test line. Goat anti-mouse immunoglobulin (Goat anti-mouse IgG) as a ligand of the control line was dispensed and dried onto the control line.

B. Conjugate Pad Fabrication

A first conjugate solution containing colloidal gold nanoparticles and anti-I-hCG antibody (Monoclonal anti-hCG) binding to the colloidal gold nanoparticles was prepared. A second conjugate solution containing colloidal gold nanoparticles and anti-βcf hCG antibody (Monoclonal anti-βcf hCG) binding to the colloidal gold nanoparticles was prepared. Further, a third conjugate solution containing colloidal gold nanoparticles and mouse immunoglobulin (Mouse IgG) binding to the colloidal gold nanoparticles was prepared. The first conjugate solution, the second conjugate solution, and the third conjugate solution were dispensed onto a pretreated conjugate pad and completely dried. Then, the pad was cut into an appropriate size.

C. Sample Pad Production

A sample pad was sufficiently soaked with a sample pad pretreatment solution containing a buffer and a preservative and completely dried, and then cut into an appropriate size.

D. Fabrication of Absorbance Pad

A wicking pad in a dry state was cut to an appropriate size.

E. Preparation of Immunochromatography Strip

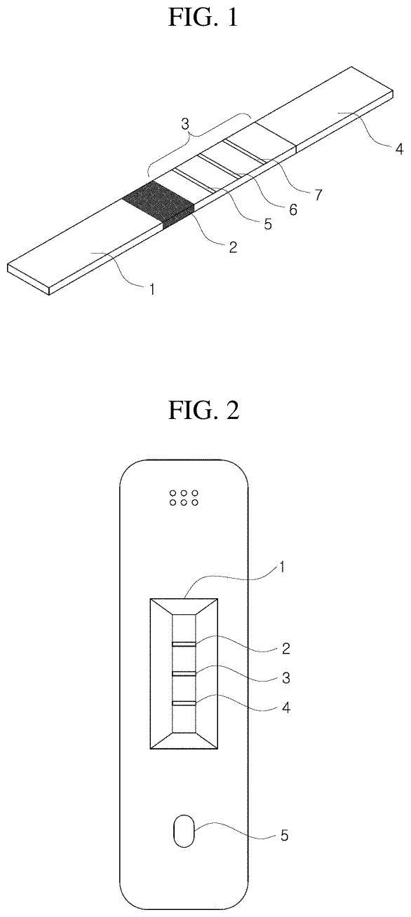

We assembled the membrane, conjugate pad, sample pad and absorbing pad prepared in each of the above steps as shown in FIG. 1.

That is, the sample pad is attached to one end of the conjugate pad in an overlapping manner One end of the detection pad is attached to the other end of the conjugate pad in an overlapping manner The other end of the detection pad and one end of the absorption pad were attached to each other in an overlapping manner

In FIG. 1, each of reference numerals are as follows.

1: sample pad

2: conjugate pad

3: nitrocellulose membrane

4: absorbent pad

5: first test line having I-hCG antibody fixed thereto

6: second test line having βcf hCG antibody fixed thereto

7: control line having goat anti-mouse immunoglobulin fixed thereto

F. Device Assembly

We placed the prepared immunochromatography strip for pregnancy diagnosis on a plastic lower device at a strip fixing position thereof. Then, we inserted a combination of the strip and the lower device into an upper device with an sample inlet and a result checking window. Thus, an assembled device was obtained.

### Present Example 2

### Analysis using Immunochromatography Strip based on I-hCG and βcf hCG Concentrations

I-hCG and βcf hCG standard substances were spiked into urine of a non-pregnant woman to prepare a mixed standard sample and a test was carried out for the sample. The prepared standard sample solution was dispensed by 100 μl into a device sample inlet and moved along the strip. Then, we checked a result after at least 3 minutes and at most 10 minutes. The concentrations of I-hCG and βcf hCG of the prepared standard sample solution are as follows (Table 1).

TABLE 1

| | | | | | Hook effect | |
|---|---|---|---|---|---|---|
| | | Experi- | Experi- | Experi- | Experi- | Experi- |
| | Cut-off | ment | ment | ment | ment | ment |
| | Value | Group 1 | Group 2 | Group 3 | Group 4 | Group 5 |
| I-hCG | ≥25 | 25 | 100 | 1,000 | 212,000 | 800,000 |
| (mIU/mL) | | | | | | |
| βcf hCG | ≥1 | 1 | 10 | 100 | 10 | 500 |
| (pmol/mL) | | | | | | |

As shown in FIG. 3, as the concentration of I-hCG in the sample increases, a signal strength of the gold particle of the test line gradually increases. Thus, the strongest intensity was induced from Experiment Group 3 having a concentration of 1,000 mIU/mL of I-hCG. In Experiment Groups 4 and 5 with I-hCG concentrations of 212,000 mIU/ml and 800,000 mIU/ml respectively, the first test line for detecting I-hCG had a very weak color or a non-detection result due to a hook phenomenon. However, in the second test line for detecting βcf hCG, βcf hCG could be detected regardless of the hook phenomenon due to an increase in I-hCG concentration in the sample (FIG. 3).

### Present Example 3

### False Negative Evaluation Based on High Concentration βcf hCG

Comparative evaluation between immune devices from three third-party domestic companies and a device of the inventive concept was performed for false negative evaluation due to the interference phenomenon by βcf hCG. The evaluation method was based on a screening of Nerenz et al. (Nerenz et al., Clinical chem, 2014, Vol. 60: 4, 667-674).

A first sample formed by mixing I-hCG 0.5 pmol/mL (about 200 mIU/mL) with the urine of a non-pregnant woman, a second sample containing a mixture of βcf hCG 50 pmol/mL and the urine of a non-pregnant woman, and a third sample formed by mixing I-hCG 0.5 pmol/mL and βcf hCG 500 pmol/mL with the urine of non-pregnant women were used and were determined visually.

When applying the second βcf hCG 50 pmol/mL based sample to the devices, and applying the third I-hCG 0.5 pmol/mL and βcf hCG 500 pmol/mL based sample thereto, the devices from the third-party companies did not exhibit a color band or had a weak color signal, thereby leading to a false negative result. This implies a possibility of the false negative determination for the actual sample. However, the immune device (ADTech) in accordance with the inventive concept exhibited a color band for all three samples, thereby to solve the problem of the false negative result (FIG. 4).

### Present Example 4

### Comparative Experiment

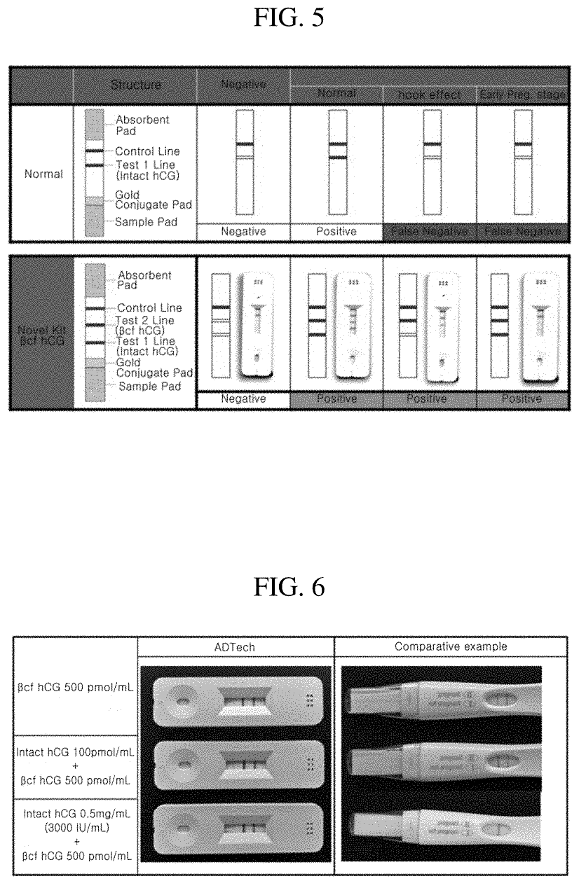

A detection region of a general pregnancy diagnostic tester has one test line and one control line. Thus, an user diagnoses a positive pregnancy when the two lines have a color. Therefore, there is a possibility of false negatives due to the hook phenomenon caused by the high concentration I-hCG or the false negatives due to the interference effect of βcf hCG in the early stage of pregnancy. On the other hand, the immune strip of the inventive concept has a separate test line for detecting βcf hCG. Thus, in spite of the hook phenomenon caused by the high concentration I-hCG or the interference effect of βcf hCG increasing in the early stage of pregnancy (since LMP 2 weeks), the separate test line may detect βcf hCG to determine that the pregnancy is positive, thus solving the false negative problem that otherwise occurs in early and late pregnancy (FIG. 5).

Further, the pregnancy test kit according to the inventive concept and the pregnancy test kit (Comparative Example 1) of C&D company were compared to each other. Comparative Example 1 showed a positive response in one test line when a high concentration of βcf hCG was applied thereto. This demonstrates that Comparative Example 1 shows no hook effect due to the βcf hCG interference. However, when Comparative Example 1 was treated with a high concentration of I-hCG (about 3,000 IU/mL) and βcf hCG at the same time, one test line showed mutual interference, leading to the false negative. In contrast, in the kit of the inventive concept characterized by the separate I-hCG and βcf hCG test lines, the Bcf hCG test line was colored without receiving a mutual interference phenomenon in the high concentration of I-hCG. Thus, the pregnancy was determined to be positive (Table 2 and FIG. 6).

TABLE 2

| | Result | |
|---|---|---|
| | Immune device of the | Comparative |
| | inventive concept | Example 1 |
| βcf hCG 500 pmol/mL | Positive | Positive |
| Intact hCG 100 mIU/mL + | Positive | Positive |
| βcf hCG 500 pmol/mL | | |
| Intact hCG 0.5 mg/mL (3,000 | Positive | Negative |
| IU/mL) + βcf hCG 500 | | (Hook effect) |
| pmol/mL | | |

The immunochromatography strip according to an embodiment of the inventive concept further includes the test line for detecting βcf hCG in addition to the test line for detecting I-hCG, such that the false negative caused by the hook phenomenon due to the high concentration of I-hCG and/or the interference of βcf hCG is minimized to enable accurate pregnancy diagnosis.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. An immune device for pregnancy diagnosis, the device comprising:
a sample region for receiving a test sample to be analyzed;
a conjugate region connected to the sample region and including an anti-I-hCG antibody conjugated with a probe and an anti-βcf hCG antibody conjugated with a probe;
a signal detection region connected with the conjugate region, which is connected with the sample region, wherein the signal detection region includes a first test line having an anti-I-hCG antibody immobilized to the first test line, a second test line having an anti-βcf hCG antibody immobilized to the second test line, and a control line; and
a wicking region located downstream of the signal detection region, wherein the wicking region absorbs the test sample for which a signal detection reaction has terminated.

2. The immune device of claim 1, wherein the probe includes at least one selected from a group consisting of gold nanoparticles, silver nanoparticles, quantum dot nanoparticles, carbon nanoparticles, latex beads/fluorescent nanoparticles, cellulose nanoparticles, magnetic nanoparticles, silica nanoparticles, polymer beads, a fluorescent substance (fluorescein), a luminescent substance, a dye, and a protein.

3. The immune device of claim 1, wherein the signal detection region includes one selected from a group consisting of nitrocellulose, cellulose, polyethylene, polyethersulfone, and nylon.

4. The immune device of claim 1, wherein the wicking region includes a porous support and an absorbent dispersed in pores of the porous support or adsorbed or coated on a fiber of the porous support.

5. The immune device of claim 1, wherein the immune device determines that a pregnancy is present when the control line and the first test line, the control line and the second test line or the control line and the first and second test lines emit a color.

6. A method for providing information for pregnancy diagnosis, the method comprising:
applying a test sample to the immune device of claim 1, such that I-hCG and βcf hCG in the sample react with the anti-I-hCG antibody conjugated with the probe and the anti-βcf hCG antibody conjugated with the probe;
checking the reaction of the I-hCG and βcf hCG in the sample using the first test line and the second test line, wherein presence of each of the I-hCG and βcf hCG is detected based on a signal resulting from presence of a sandwich complex in each of the first and second test lines; and
determining that a pregnancy is present when the control line and the first test line, the control line and the second test line or the control line and the first and second test lines emit a color.
